(19) United States
(12) Patent Application Publication
(10) Pub. No.: US 2024/0285316 A1
(43) Pub. Date: Aug. 29, 2024

(54) THORACOLUMBAR PLATING SYSTEM AND RELATED METHODS

(71) Applicant: GLOBUS MEDICAL, INC., AUDUBON, PA (US)

(72) Inventors: Colm McLaughlin, Glenside, PA (US); Julia Gambogi, Eagleville, PA (US)

(21) Appl. No.: 18/173,467

(22) Filed: Feb. 23, 2023

(51) Int. Cl. A61B 17/70; A61B 17/80

(52) U.S. Cl. CPC A61B 17/7059; A61B 17/8042

(57) ABSTRACT

A thoracolumbar plating system includes a plate body, screw holes configured to receive bone screws, and one or more blocking members configured to allow passage of the bone screws into the plate body and to lock the bone screws in the plate body after passage. The plating system allows angulation of the bone screws while position in the plate body prior to fixation to a vertebral body. The plating system also allows rotation of one or more blocking members from an unlocked position to a locked position. The plate body may include three screw holes, four screw holes, or more. The blocking members may block a single bone screw or multiple bone screws. Components of the plating system may include strain sensors to monitor forces exerted on the components and a transmitter box to send communications relating to the exerted forces.

### FIELD

The present invention relates to stable fixation of spine segments, allowing for fusion in, e.g., skeletally mature patients. More particularly, the invention relates to a bone fixation device that can be affixed to vertebrae of a spine to provide reduction (or enlargement) capabilities and allow for fixation in the treatment of various conditions, including, e.g., spondyloslisthesis, degenerative disc disease, fracture, dislocation, spinal tumor, failed previous fusion, and the like, in the spine. The invention also relates to a method for delivering and implanting the bone fixation plating device.

### BACKGROUND

Bones and bony structures are susceptible to a variety of weaknesses that can affect their ability to provide support and structure. Weaknesses in bony structures can have many causes, including degenerative diseases (e.g., degenerative disc diseases), tumors, fractures, dislocations and failed previous fusions. Some of these weaknesses can cause further conditions such as spondylolisthesis wherein bony structures slip out of their proper position.

In some cases of spinal surgery, it is known to use bone fixation plating devices (e.g., bone plate systems and rod and screw systems) to improve the mechanical stability of the spinal column and to promote the proper healing of injured, damaged or diseased spinal structures. Typically, corrective surgery can entail the removal of damaged or diseased tissue, a decompression of one or more neural elements, followed by the insertion of an interbody implant or bone graft for the purposes of a fusion or disc arthroplasty. In cases where spinal fusion is the desired surgical outcome, the surgery can often include implanting a bone plate or rod and screw system in order to immobilize adjacent vertebral bones to expedite osteogenesis across the vertebral segments.

Plates have been used frequently for stabilization in the thoracolumbar spine. Thoracolumbar plating is often used in conjunction with spinal fusion to add stabilization to the segment. However, vertebrae and different surfaces on those vertebrae have varying shapes which causes variation in the desirable plate contour and screw trajectory. Improper placement and fit of a plate onto the vertebral bodies can weaken fixation and can also cause damage to the surrounding soft tissue and vasculature.

Additional issues include intra-operative movement in a thoracolumbar plate during intra-operative placement and screw prep and to assist in the reduction of migration for the implant's life. There also exists a need for means of data collection on the forces in the segment allow for aid in placement intra-operatively and for patient monitoring post-operatively.

Accordingly, there is a need to improve on bone fixation plating devices including a need to provide a rigid construct of plates and screws able to be placed anteriorly, laterally, or anterior-laterally onto the spine and match the natural curvature of a vertebral body and provide stability for fusion. There is also a need to address the problem of fitting anterior thoracolumbar plates with the vertebral bodies throughout the implant's life. Therefore, there is a need for an improved plating system that will be fixated to the anterior, anterolateral, or lateral aspect of the vertebral bodies.

### SUMMARY

In accordance with the present disclosure, a thoracolumbar plating system including a bone plate having a plate body including a plurality of screw holes and a plurality of blocking members and a plurality of bone screws, each bone screw received in one of the plurality of screw holes. Each blocking member rotates from an unlocked position, allowing one of the plurality bone screws to be received in one of the screw holes, to a locked position, preventing the bone screw from backing out of the plate body.

In accordance with the present disclosure, a thoracolumbar plating system including a bone plate having a plate body including a plurality of screw holes and one or more blocking members and a plurality of bone screws, each bone screw received in one of the plurality of screw holes. The one or more blocking members rotate from an unlocked position, allowing multiple bone screws to be received in one of the screw holes, to a locked position, preventing multiple bone screws from backing out of the plate body.

### BRIEF DESCRIPTION OF THE DRAWINGS

The following detailed description and examples are provided for the purpose of non-exhaustively describing some, but not necessarily all, examples or embodiments of the disclosure, and shall not limit the scope of the disclosure in any way.

FIG. 1 illustrates a bone plate according to some exemplary embodiments;

FIG. 2 illustrates a bone plate according to some exemplary embodiments;

FIG. 3 illustrates the bone plate of FIG. 1 with an implant according to some exemplary embodiments;

FIG. 4 illustrates the bone plate of FIG. 2 with an implant according to some exemplary embodiments;

FIGS. 5 and 6 illustrate a bone plate with a bone screw according to some exemplary embodiments;

FIGS. 7 and 8 illustrate a blocking member of a bone plate accordingly to some exemplary embodiments;

FIG. 9 illustrates the bone plate of FIG. 1 in an unlocked position according to some exemplary embodiments;

FIG. 10 illustrates the bone plate of FIG. 1 in an locked position according to some exemplary embodiments;

FIG. 11 illustrates the bone plate of FIG. 2 in an unlocked position according to some exemplary embodiments;

FIG. 12 illustrates the bone plate of FIG. 2 in a locked position according to some exemplary embodiments;

FIGS. 13 and 14 illustrate a bone plate according to some exemplary embodiments;

FIGS. 15A-15F illustrate bone plates according to some exemplary embodiments;

FIG. 16 illustrates a bone plate secured to spine accordingly to some exemplary embodiments;

FIG. 17 illustrates a bone screw according to some exemplary embodiments;

FIGS. 18 and 19 illustrate a bone plate according to some exemplary embodiments;

FIGS. 20A and 20B illustrate a bone plate with strain sensors and transmitter boxes according to some exemplary embodiments;

FIG. 21 illustrates a bone plate with a strain sensor and transmitter box according to some exemplary embodiments;

FIGS. 22A and 22B illustrate a bone plate and a bone screw with strain sensors and transmitter boxes according to some exemplary embodiments;

FIGS. 23A and 23B illustrates a bone screw with strain sensors and transmitter box according to some exemplary embodiments;

FIGS. 24A and 24B illustrate a blocking member of a bone plate with a strain sensor and transmitter box according to some exemplary embodiments;

FIG. 25 illustrates a bone plate according to some exemplary embodiments;

FIG. 26 illustrates a bone plate according to some exemplary embodiments;

FIGS. 27 and 28 illustrate a blocking member according to some exemplary embodiments;

FIG. 29 illustrates the bone plate of FIG. 26 in an unlocked position;

FIG. 30 illustrates the bone plate of FIG. 26 in a locked position;

FIG. 31 illustrates the bone plate of FIG. 25 in an unlocked position;

FIG. 32 illustrates the bone plate of FIG. 25 in a locked position; and

FIGS. 33, 34, and 35 illustrate bone plates according to some exemplary embodiments.

### DETAILED DESCRIPTION OF THE EMBODIMENTS

It should be understood that this disclosure is not limited to the particular apparatus, methodology, protocols, and systems, etc., described herein and as such may vary. The terminology used herein is for the purpose of describing particular embodiments only and is not intended to limit the scope of the present disclosure, which is defined solely by the claims.

Described in further detail below and consistent with the principles of the present disclosure is a 4-screw and 3-screw plate design that allows for bone screw engagement for stabilization with a high level of screw trajectory variation. The 3-screw plate design allows for similar bone screw engagement for stabilization as a 4-screw plate, while reducing the amount of bone material removed or potentially damaged by screw prep and insertion for the 4th bone screw. By maintaining bone quality, there is less of a chance of subsidence and screw pullout of the plating system or other implants. Additionally, the smooth profile of the plate and the options of sacral 4-screw or 3-screw plating allow for optimal contact between the plate and vertebral body. The blocking set screw design for all of the plating options allows for the variability in screw placement while maintaining coverage of the screw head to prevent screw backout. The low-profile plates and shortened screw heads allow for minimal protrusion of the plate addressing the issue of disturbance to the surrounding vasculature.

This plating system will be fixated to the anterior, anterolateral, or lateral aspect of the vertebral bodies. The surgeon choses placement of the implant based on patient anatomy and also based on other existing instrumentation. Once placed, screw preparation (awl, tap, etc.) and screw placement will be performed which may put forces on the plate causing intra-operative movement. Additionally, throughout the implants life post-operatively there will be loads on the plate from patient movement. The primary prevention of the plate's migration is the bone screws/fixation, however the ‘ridge’ features described below that will help to prevent the migration of the plate from its placement on the spine.

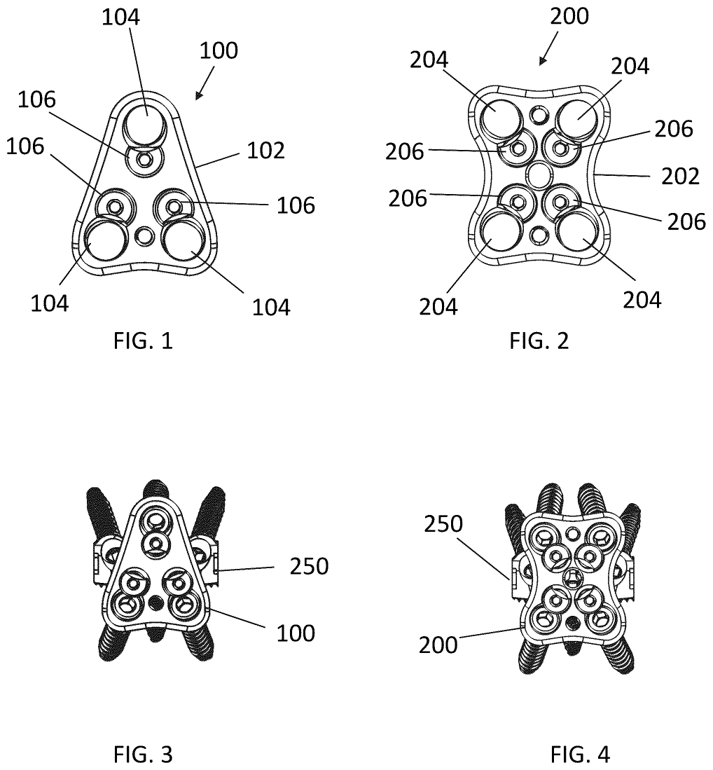

Turning now to FIGS. 1 and 2, illustrated are a 3-screw plate 100 and 4-screw plate 200 consistent with exemplary embodiments of the present disclosure. Plate 100 includes a plate body 102, a set of screw holes 104, and blocking members 106. Plate 200 includes a plate body 202, a set of screw holes 204, and blocking members 206. As shown in FIGS. 3 and 4, plates 100 and 200 can be used in conjunction with a implant 250 configured to be inserted within a disc space of patient.

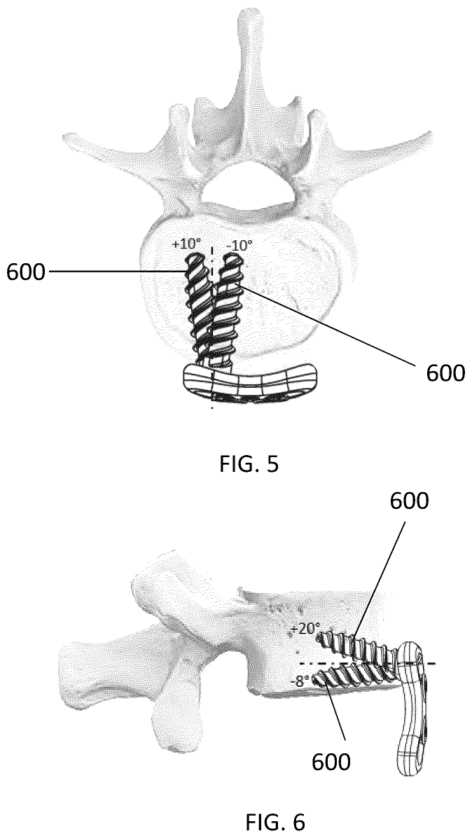

As shown in FIGS. 5 and 6, each of plates 100 and 200 allow for a high level of screw angulation in the axial plane and the sagittal plane. For example, FIG. 5 shows a bone screw 600 (described in further detail below) with medial/lateral angulation of +10° to −10° from nominal (0°) in the axial plane and FIG. 6 shows bone screw 600 with up to +20°/−8° from nominal (0°) in the sagittal plane.

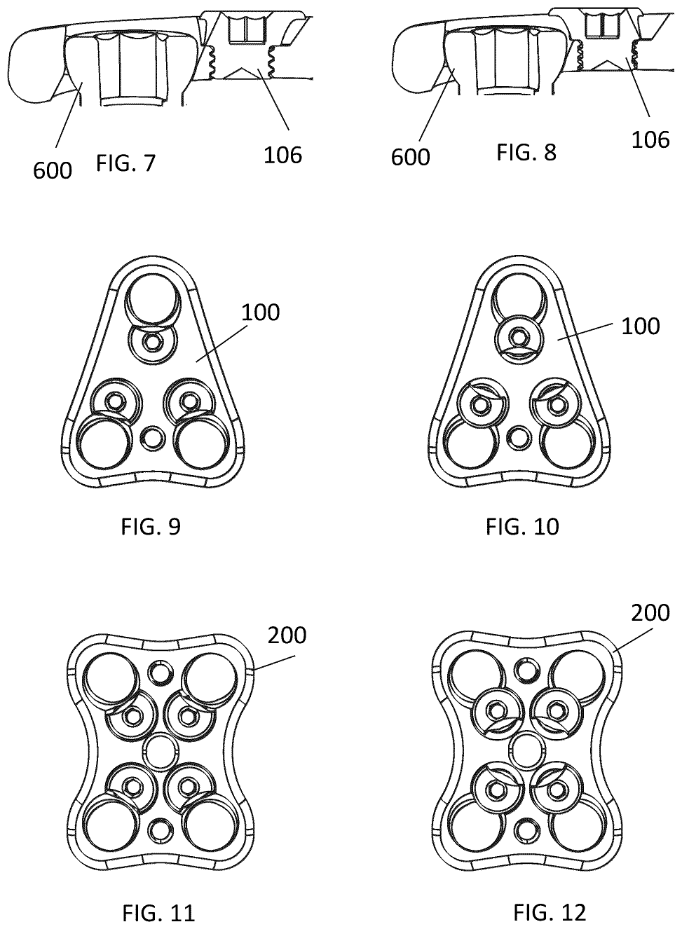

Turning now to FIGS. 7-12, blocking members 106 and 206 will be explained in further detail. Consistent with principles of the present disclosure, blocking members 106, 206 block a screw head after screw insertion, by turning blocking members 106, 206, which may be in the form of a set screw. As shown in FIGS. 7 and 8, blocking member 106 provides additional material above the screw head to prevent the screw head from backing out of its position in the plate. In FIG. 7, the blocking member is in a position to allow the screw to enter the screw hole. In FIG. 8 the blocking member is rotated from its position in FIG. 7 to lock the screw in the screw hole. FIGS. 9 and 11 show plates 100 and 200, respectively, in an unlocked position. FIGS. 10 and 12 show plates 100 and 200, respectively, in a locked position.

Blocking members 106 and 206 may be threaded blocking set screws with clearance for the bone screw to pass into the screw hole and with material removed for a cavity where the screw head will reside when the blocking set screw is blocking it from backing out. The locking position is obtained through a one-step turn of the blocking set screw which will tighten the threads so that the lock does not become unlocked.

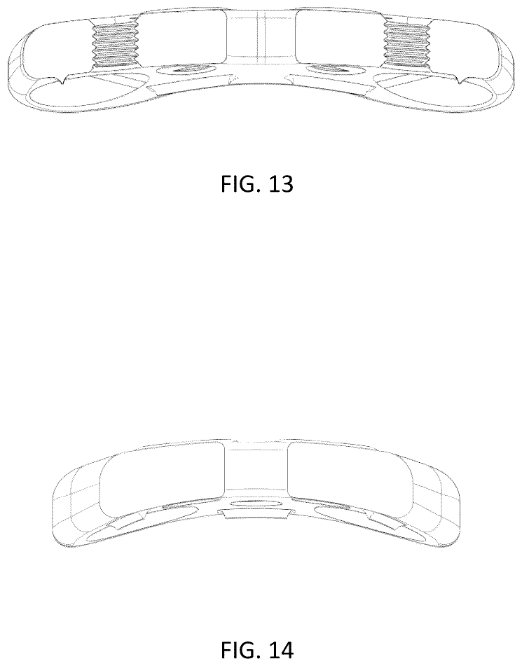

Turning to FIGS. 13 and 14, each of plates 100 and 200 may have a smooth curved surface for better contact and conformance with the vertebral body and a low-profile plate thickness that is nominally 3.5 mm.

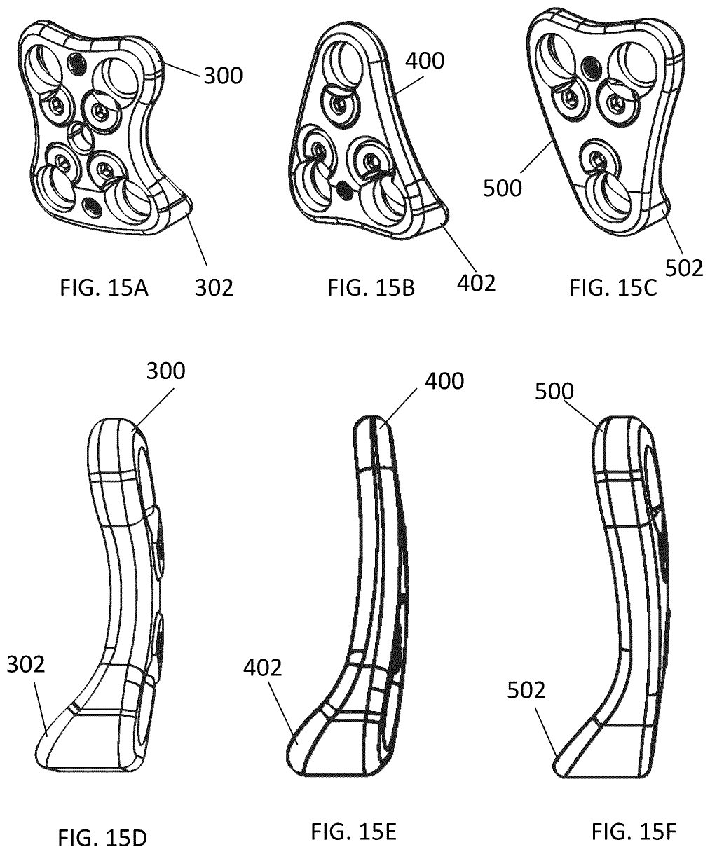

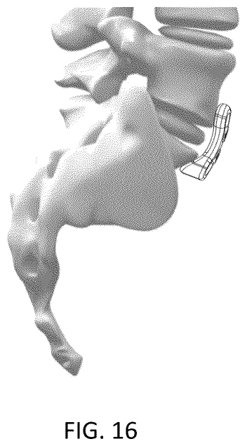

FIGS. 15A-F show alternative plates 300, 400, and 500 that are sacral plate options. These plates are a 4-screw plate or a 3-screw plate, each having a sacral bump 302, 402, and 502 that deviates from the nominal 3.5 mm thickness to a sharper curvature to match the angular anterior curvature of the sacrum on either the 1-screw side or the 2-screw side. FIG. 16 illustrates a sacral plate according to the principles of the present disclosure implanted at the L5-S1 segment.

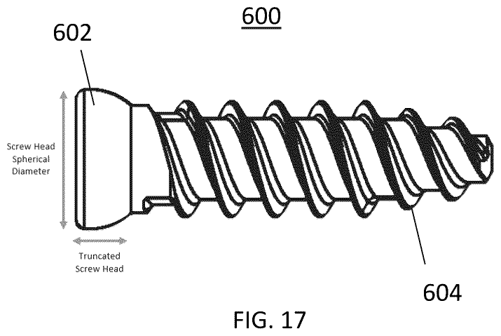

FIG. 17 shows an exemplary embodiment of bone screw 600 consistent with the principles of the present disclosure. Bone screw 600 includes a truncated screw head 602 with a large spherical diameter. This prevents screw 600 from protruding from the face of the plate while maintaining the strength of connection between screw head 602 and a plate pocket due to the wider screw head. Bone screw 600 may also have deep thread 604 to help provide fixation in the cancellous bone of the vertebral bodies. This plate system works with self-drilling and self-tapping bone screws.

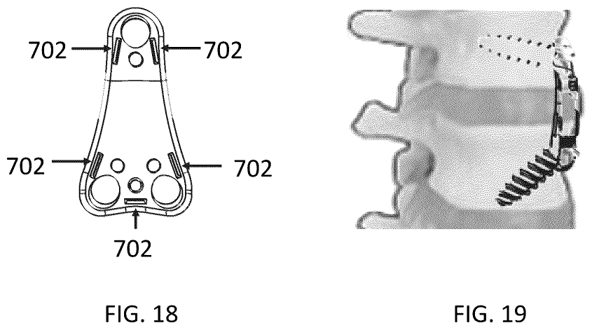

As shown in FIGS. 18 and 19, each of plates 100 and 200 may have anti-migration features. These features may be on the bottom face of the plate around the screw holes to cut into the bone of the vertebral bodies and provide additional friction to the plate surface to vertebral body face interaction. These features may be in a form of ridges 702 on the back face plate 100 has shown in FIG. 18. Plate 200 may also have similar ridges designed for the same purpose. FIG. 19, shows an exemplary bone plate with ridges engaging a spine of a patient.

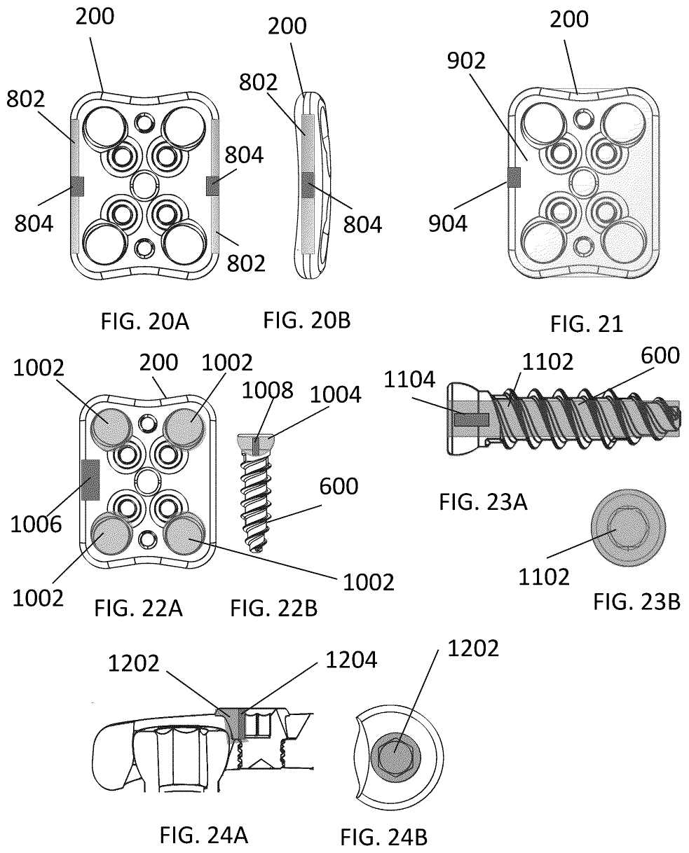

FIGS. 20A-24B illustrate exemplary embodiments of the previously described implants with strain sensors. This invention includes the possible use of sensors of different placements and orientations that allow for data collection and computation throughout the implant's life. Several possible gauge configurations are shown.

FIGS. 20A and 20B illustrates plate 200 that includes sensors 802 that determine the strain on plate 200 created by the construct between fixation to the superior vertebra and the inferior vertebra. Transmitter boxes 804 may be used to compute and communicate information.

FIG. 21 illustrates plate 200 with a sensor 902 (or a group of sensors) to determine overall forces throughout plate 200. A transmitter box 904 may be used to compute and communicate information.

FIGS. 22A-22B illustrates plate 200 with sensors 1002 and bone screw 600 with sensor 1004. These allow for information on the forces in placing the screw through the plate which could include how the thread and shanks affect the plate when being driven down and also how the screw head sits in the plate. Beyond intra-op uses, this design allows for information on screw and plate interaction forces to be gathered throughout the life of the implants. Transmitter boxes 1006 and 1008 are also included to compute and communicate the information gathered.

FIGS. 23A-23B illustrates bone screw 600 with sensors 1102 that determine the stresses and strains along the length of the screw and throughout its body. Sensor 1102 in the bone screw's hex allow for information to be collected that can be paired with a screw driver with sensing capabilities to gauge when the screw female hex or driver male hex might strip or deform. A transmitter box 1104 may be used compute and communicate information.

FIGS. 24A-B illustrate a blocking member that includes sensors 1202 on the blocking screw to be able to determine confident blocking of the bone screw intra-operatively and throughout the implant's life. This design may collect information on the placement of the bone screw in relation to the blocking screw and also the forces on the blocking screw that may come from the bone screw or other factors. Sensor 1202 in the blocking screw's hex allow for information to be collected that can be paired with a screw driver with sensing capabilities to gauge when the screw female hex or driver male hex might strip or deform A transmitter box 1204 may be used to compute and communicate information.

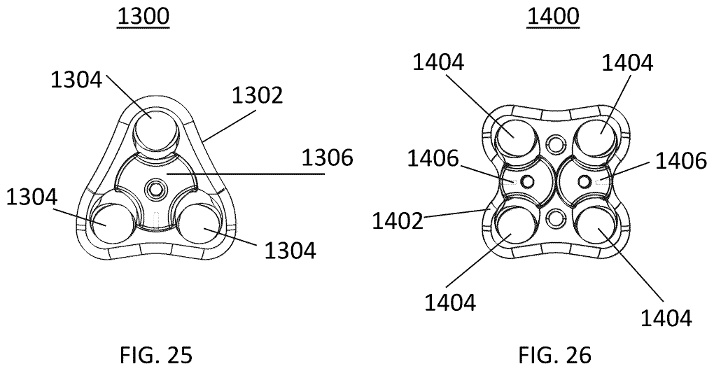

Turning now to FIGS. 25-32, alternative designs for the 3-screw and 4-screw plates are illustrated. In these embodiments, the plates have a different type of blocking member than previously described. For example, in FIG. 25, plate 1300 includes a plate body 1302, screw holes 1304, and a blocking member 1306. Here, blocking member 1306 may be moved from an locked to locked position to simultaneous lock all screws in screw holes 1304. FIG. 26 shows a plate 1400 including a plate body 1402, screw holes 1404, and blocking members 1406. Blocking members 1406 are configured to lock multiple screws in place. In other respects, blocking members 1306 and 1406 may have similar features as blocking members 106 and 206.

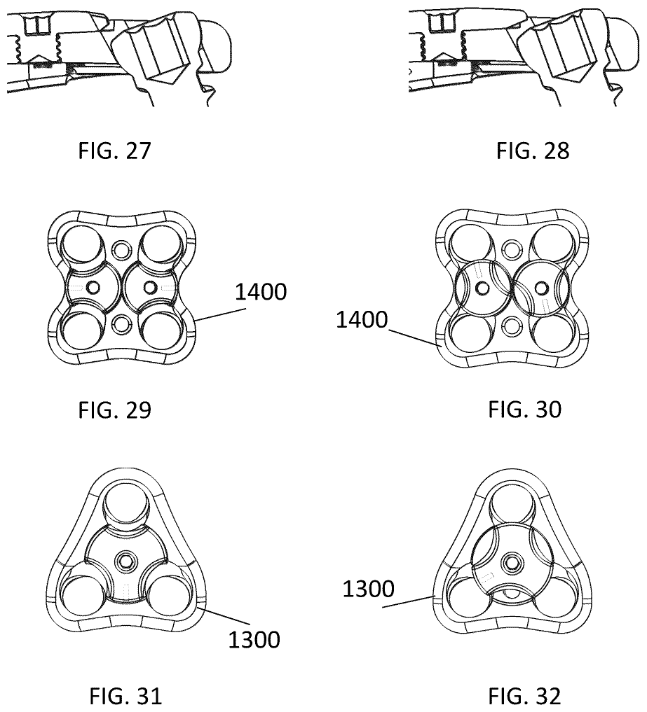

In FIG. 27, the blocking member is in a position to allow the screw to enter the screw hole. In FIG. 28 the blocking member is rotated from its position in FIG. 27 to lock the screw in the screw hole. FIGS. 29 and 31 show plates 1400 and 1300, respectively, in an unlocked position. FIGS. 30 and 32 show plates 1400 and 1300, respectively, in a locked position.

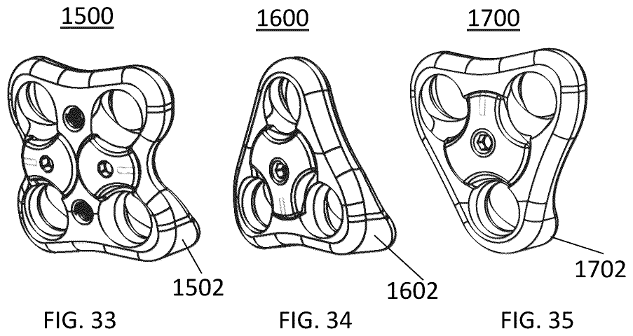

Similarly as discussed before, FIGS. 33-35 show alternative plates 1500, 1600, and 1700 that are sacral plate options. These plates are a 4-screw plate or a 3-screw plate, each having a sacral bump 1502, 1602, and 1702 that deviates from the nominal thickness of the plate to a sharper curvature to match the angular anterior curvature of the sacrum on either the 1-screw side or the 2-screw side.

All designs listed above can be included into the plating system individually or with any combination of designs working together within the system. For all potential design configurations, a reading could be taken intraoperatively to get information on how the construct has been fit on the spinal segment, and then post-operatively readings can be taken to gather information on the movement/stress/strain/forces of the segment as the fusion occurs and beyond. The information collected would be able to be computed and accessed from an implanted system through the anatomy without additional surgery needed.

Advantages of the principles described herein include a close fit to the spinal anatomy with the described plating system to allow for a stronger construct for spinal stability in varying approaches and vertebral bodies. The variability in screw trajectories and screw angulation, and the slim profile with the option of a sacral bump all contribute to a better fit. In addition, the fit of the plate can be monitored throughout the life of the implant through a strain measuring sensor able to output and/or computed data.

Screw angulation and trajectory allow for placing shorter plates and being able to avoid objects that might get in the way of the screw path which may include existing implants, future posterior fixation, access issues, or difficult anatomy. This also allows for better bone contact and fixation.

The slim profile of the plate aids in reducing the protrusion from the spine, which is beneficial since there is sensitive vasculature anterior to the spine in this area. Additionally, the sacral bump that matches the curvature of the sacrum is offered in the form of a 4-screw plate and a 3-screw plate with the bump either on the 1-screw side or the 2-screw side.

The construct is secured by having blocking screws that cover the bone screw no matter the angulation or trajectory to help fixation and have a stable construct.

The features on the back face of the plates prevents migration of the plate on the vertebral bodies intra-operatively and post-operatively. This may help reduce operating time as the surgeon may need to re-position the plate if there is migration intra-operatively. These features can also be a benefit to the plate's function through the life of the implant.

The data collection and output of measurements in the plate and bones screws allow for information on the fit and strength of the construct. The surgeon may be able to drive their choices in implant size and placement based on the forces seen intra-operatively. The intraoperative information can also help warn the surgeon and prevent implant breakage and stripping/deforming of the screws and drivers. The sensors have the capability of transmitting and computing data throughout the implants life which allows for monitoring potential damage to the implants. Since thoracolumbar plates are often used to help stabilize the spinal segment by fixating to the superior and inferior vertebrae, the data available would provide valuable information on the movements of the segment, including: potential indications of fusion based on movement seen in flexion/extension, warnings of increased strain indicating subsidence, overall anatomical movements and forces able to be collected for better understanding of the spine.

Other advantages include the ability to include a short plate option which does not have space for individual blocking screws, but can be confidently blocked with a multi-blocking screw. Including a blocking screw that covers multiple bone screws also adds the advantage of having fewer surgical steps. Additionally, the sacral bump that matches the curvature of the sacrum is offered in the form of a 4-screw plate and a 3-screw plate with the bump either on the 1-screw side or the 2-screw side. Having a variety of options for plate profile allows for a closer fit to patient anatomy.

It is intended that all matter contained in the above description or shown in the accompanying drawings shall be interpreted as illustrative only and not limiting. Changes in detail or structure may be made without departing from the spirit of the disclosure as defined in the appended claims.

What is claimed is:

1. A thoracolumbar plating system comprising:
a bone plate having a plate body including a plurality of screw holes and a plurality of blocking members;
a plurality of bone screws, each configured to be received in one of the plurality of screw holes;
wherein each blocking member is configured to rotate from an unlocked position, allowing one of the plurality bone screws to be received in one of the screw holes, to a locked position, preventing the bone screw from backing out of the plate body.

2. The thoracolumbar plating system of claim 1, wherein each of the bone screws is allowed to angulate within one of the plurality of screw holes in the axial plane and the sagittal plane.

3. The thoracolumbar plating system of claim 2, wherein each of the bone screws is configured to have medial/lateral angulation of +10° to −10° from a nominal position of 0° in the axial plane.

4. The thoracolumbar plating system of claim 2, wherein each of the bone screws is configured to angulation up to +20°/−8° from a nominal position of 0° in the sagittal plane.

5. The thoracolumbar plating system of claim 1, wherein each blocking member is configured to rotate from the unlocked position to the locked position.

6. The thoracolumbar plating system of claim 5, wherein each of the blocking members is a set screw.

7. The thoracolumbar plating system of claim 1, wherein the plate body and/or the bone screw have at least one strain sensor to monitor exerted forces and a transmitter box to send out information related to the exerted forces.

8. The thoracolumbar plating system of claim 1, wherein the plurality of screw holes is three screw holes.

9. The thoracolumbar plating system of claim 1, wherein the plurality of screw holes is four screw holes.

10. The thoracolumbar plating system of claim 1, wherein the plate body includes a sacral bump configured to engage an L5-S1 segment.

11. A thoracolumbar plating system comprising:
a bone plate having a plate body including a plurality of screw holes and one or more blocking members;
a plurality of bone screws, each configured to be received in one of the plurality of screw holes;
wherein the one or more blocking members are configured to rotate from an unlocked position, allowing multiple bone screws to be received in one of the screw holes, to a locked position, preventing multiple bone screws from backing out of the plate body.

12. The thoracolumbar plating system of claim 1, wherein each of the bone screws is allowed to angulate within one of the plurality of screw holes in the axial plane and the sagittal plane.

13. The thoracolumbar plating system of claim 2, wherein each of the bone screws is configured to have medial/lateral angulation of +10° to −10° from a nominal position of 0° in the axial plane.

14. The thoracolumbar plating system of claim 2, wherein each of the bone screws is configured to angulation up to +20°/−8° from a nominal position of 0° in the sagittal plane.

15. The thoracolumbar plating system of claim 1, wherein each blocking member is configured to rotate from the unlocked position to the locked position.

16. The thoracolumbar plating system of claim 5, wherein each of the blocking members is a set screw.

17. The thoracolumbar plating system of claim 1, wherein the plate body and/or the bone screw have at least one strain sensor to monitor exerted forces and a transmitter box to send out information related to the exerted forces.

18. The thoracolumbar plating system of claim 1, wherein the plurality of screw holes is three screw holes.

19. The thoracolumbar plating system of claim 1, wherein the plurality of screw holes is four screw holes.

20. The thoracolumbar plating system of claim 1, wherein the plate body includes a sacral bump configured to engage an L5-S1 segment.
